(19) United States
(12) Patent Application Publication
(10) Pub. No.: US 2023/0271006 A1
(43) Pub. Date: Aug. 31, 2023

(54) Method For Treating Living Body Using Electrical Stimulator

(71) Applicants: SUMIDA CORPORATION, Tokyo (JP); TOYO UNIVERSITY, Tokyo (JP)

(72) Inventors: Tsunaki KANEKO, Natori City (JP); Masaki SAITO, Natori City (JP); Thitaporn DETMOD, Natori City (JP); Kenji TERAO, Natori City (JP); Masafumi OHSAKO, Asaka-shi (JP); Xueqian ZENG, Asaka-shi (JP)

(21) Appl. No.: 18/169,319

(22) Filed: Feb. 15, 2023

(30) Foreign Application Priority Data
Feb. 25, 2022 (JP) 2022-027532

(51) Int. Cl. A61N 1/36

(52) U.S. Cl. CPC A61N 1/36014; A61N 1/36034

(57) ABSTRACT

Provided is a method for treating a living body using an electrical stimulator including a base wire having a core wire and an outer winding wire wound around the core wire. An annulus is formed by winding the base wire in a loop shape. A first end of the core wire is electrically connected to a first end of the outer winding wire. A second end of the core wire is connected to a first terminal of an external circuit. A second end of the outer winding wire is connected to a second terminal of the external circuit. The method includes holding the living body or a part of the living body of a subject in the annulus and generating an alternating current in the external circuit for a therapeutically effective time period to apply an electrical stimulation to the living body or the part of the living body.

### CROSS-REFERENCE TO RELATED APPLICATIONS

This application claims priority to Japanese Patent Application No. 2022-27532 filed in Japan on Feb. 25, 2022, which is hereby expressly incorporated by reference in its entirety.

### BACKGROUND

### 1. Technical Field

The present invention relates to a method for operating an electrical stimulator and to a method for treating a living body using the electrical stimulator.

### 2. Related Art

Conventionally, a therapy that utilizes an electrical stimulation is used for osteoarthritis of the knee in clinical practice. For example, Japanese Patent Publication Number 2017-507751 discloses a method and device for accelerating bone growth and tissue healing by applying an electric current to a bone and the soft tissue adjacent to the bone via a partially insulated screw. Japanese Patent Laid-Open Number 2019-146976 discloses a small-sized thin implantable electroacupuncture (EA) device having a coin size and an improved electrode that is adopted in operating the device.

However, in these therapies, surgery is performed on a patient, or an acupuncture needle is inserted to the affected part through the skin and hence, extremely shocking stimulations are applied to the patient. Further, these shocking stimulations may cause the skin and muscles of the patient to contract, thus lowering the effect of treatment.

As a treatment method that utilizes a percutaneous electrical stimulation, Japanese Patent Publication Number 2017-503612 discloses a method and device for treating, by using an electrical stimulation, fibromyalgia and other neurological diseases including central pain, central sensitization, and abnormal connectability of the neural circuit network of the brain. However, by percutaneous electrical stimulation, an electric current cannot reach the deep layer of an affected part due to an influence of the skin, subcutaneous fat, a body fluid and the like and hence, an ideal effect of treatment cannot be obtained.

A non-contact space electric field generation device is disclosed that generates a vector potential without generating a magnetic field, thus generating a linear electric field to work outside (see International Publication Number WO2015/099147, for example). There is a report that an electrical stimulator that can achieve a shorter treatment time, less burden or less damage on a living body, and an easier attachment onto the patient is manufactured with this principle, and this electrical stimulator can treat personal injuries or damage to a human body, such as a bone fracture, a bone disorder, such as osteoporosis, and lumps such as tumors or neoplasms (see Japanese Patent Laid-Open Number 2020-58523, for example).

It is suggested that the electrical stimulator disclosed in Japanese Patent Laid-Open Number 2020-58523 can be easily attached onto the patient with less burden or less damage on a living body, thus applying an electrical stimulation to a predetermined human body part, for example, a part of the bone, certainly leading to earlier recovery of bone fracture or suppression of progression of osteoporosis or the like. However, neither a specific method for operating the electrical stimulator nor other diseases or injuries to be treated by the electrical stimulator is clearly disclosed.

### SUMMARY

In view of the above, it is an object of the present disclosure to clarify the effects of an electrical stimulation by a vector potential generation device, such as that disclosed in Japanese Patent Laid-Open Number 2020-58523, on various diseases, particularly on the articular cartilage, and to provide a method for operating the electrical stimulator and a method for treating a living body using the electrical stimulator.

The present disclosure attempts to solve the above problems. It is found that an electrical stimulation caused by the generation of a vector potential can improve various functions of the living body. It is particularly found that by applying an electrical stimulation to the articular cartilage, it is possible to maintain the normal structure and the function of the articular cartilage. The present disclosure includes the following embodiments.

(1) A method for operating an electrical stimulator that includes a base wire configured with a core wire having an insulating film, and an outer winding wire wound around the core wire with the core wire serving as a winding axis, wherein an annulus is formed by winding the base wire in a loop shape, a first end of the core wire is electrically connected to a first end of the outer winding wire, a second end of the core wire is connected to a first terminal of an external circuit, and a second end of the outer winding wire is connected to a second terminal of the external circuit, the method including: holding a living body or a part of the living body in the annulus; and generating an alternating current in the external circuit for a therapeutically effective time period to apply an electrical stimulation to the living body or the part of the living body.
(2) The method for operating the electrical stimulator of (1), in which the part of the living body is a stem cell, or a bone, a joint, or a ligament of a subject.
(3) The method for operating the electrical stimulator of (1) or (2), in which the part of the living body is a knee joint of a subject in need of treatment for osteoarthritis of the knee.
(4) The method for operating the electrical stimulator of any one of (1) to (3), in which a frequency of the alternating current is 10 to 50 kHz.
(5) The method for operating the electrical stimulator of any one of (1) to (4), in which a frequency of the alternating current is approximately 20 kHz.
(6) The method for operating the electrical stimulator of any one of (1) to (5), in which the therapeutically effective time period is at least 30 minutes/day.
(7) The method for operating the electrical stimulator of any one of (1) to (6), in which the alternating current is applied by the external circuit such that an intensity of an electric field in the annulus is 0.17 to 0.27 V/m.
(8) The method for operating the electrical stimulator of any one of (1) to (7), in which the alternating current is applied by the external circuit such that an intensity of an electric field in the annulus is approximately 0.22 V/m.
(9) The method for operating the electrical stimulator of any one of (1) to (8), in which a plurality of layers of the annulus are concentrically formed.

According to the present invention, it is possible to apply a stimulation by an electrical field to the deep layer of the living body placed in the annulus of the electrical stimulator without applying a shocking stimulation to the living body, by an electrical stimulation generated using the vector potential generation device.

### BRIEF DESCRIPTION OF THE DRAWINGS

FIG. 1 is a schematic view for explaining a base wire forming an electrical stimulator used in a method of the present disclosure.

FIG. 2 is a schematic view for explaining the electrical stimulator that uses the base wire shown in FIG. 1.

FIG. 3 is a schematic view of an experimental vector potential generation device used in examples.

FIG. 4 shows optical microphotographs of toluidine blue stained non-decalcified resin-embedded ground samples after one, two, and three weeks of the tibias of rats in a hindlimb suspension group (HS group), in a hindlimb suspended and VP stimulated group (VP group), and in a normally bred group (CO group) in an example 1.

FIG. 5 shows enlarged images of the articular cartilages shown in FIG. 4.

FIG. 6 shows the results of measurement of the thicknesses of the articular cartilages using the non-decalcified resin-embedded ground samples after one, two, and three weeks of the tibias of the rats in the hindlimb suspension group (HS group), in the hindlimb suspended and VP stimulated group (VP group), and in the normally bred group (CO group) in the example 1.

FIG. 7 shows optical microphotographs of immunostained decalcified paraffin sections after one, two, and three weeks of the tibias of the rats in the hindlimb suspension group (HS group), in the hindlimb suspended and VP stimulated group (VP group), and in the normally bred group (CO group) in the example 1.

FIG. 8 shows optical microphotographs of safranin-O stained decalcified paraffin sections after one, two, and three weeks of the tibias of the rats in the hindlimb suspension group (HS group), in the hindlimb suspended and VP stimulated group (VP group), and in the normally bred group (CO group) in the example 1.

FIG. 9 shows optical microphotographs of toluidine blue stained non-decalcified resin-embedded ground of the hindlimbs of rats in a hindlimb suspension group (HS group), in a hindlimb suspended and VP stimulated group (VP group), and in a normally bred group (CO group) in an example 2.

FIG. 10 shows optical microphotographs of toluidine blue stained non-decalcified resin-embedded ground sections of the hindlimbs of the rats in the hindlimb suspension group (HS group), in the hindlimb suspended and VP stimulated group (VP group), and in the normally bred group (CO group) in the example 2.

FIG. 11 shows scanning electron microphotographs of the deep layers of the articular cartilages of the rats in the hindlimb suspension group (HS group), in the hindlimb suspended and VP stimulated group (VP group), and in the normally bred group (CO group) in the example 2.

### DESCRIPTION OF EXEMPLARY EMBODIMENTS

Embodiments of the present disclosure will be explained with reference to drawings. The embodiments described hereinafter do not limit the invention according to the claims, and all of various elements and combinations of the elements described in the embodiments are not necessarily essential to means provided by aspects of the invention.

### Definition

The terms “treat”, “treating” or “treatment”, as used herein, refer to a therapeutic treatment, to a prophylactic (or preventive) treatment, or to both a therapeutic treatment and a prophylactic (or preventive) treatment, wherein the object is to prevent, reduce, alleviate, and/or slow down (lessen) one or more of the symptoms or manifestations of the bone or joint related disorders, in a subject in need thereof. The term “subject” or “patient” means any subject for which treatment is desired, such as humans, cattle, dogs, cats, guinea pigs, rabbits, rats, mice, horses, chickens, etc. Most preferably, the subject is a human. In addition, the term “electrical stimulation” refers to electrical stimulation obtained by holding a living body or part thereof in an electric field applied by a vector potential, which stimulation is characterized by the application of a non-contact, uniform electric field to the living body or part thereof. Furthermore, the term “therapeutically effective time period” refers to the time (minutes or hours) in which prevention, reduction, mitigation or alleviation of one or more of the bone or joint related disorders can be achieved without causing significant negative or harmful side effects to the subject requiring treatment.

### <Electrical Stimulator>

An electrical stimulator 1 used in a method of the present disclosure has a base wire 10 configured with a core wire 21, an insulating film on the core wire 21, and an outer winding wire 22 wound around the core wire 21 without any clearance between the core wire 21 and the outer winding wire 22, the core wire 21 serving as a winding axis. The base wire 10 forms an annulus 20 (annular member 20) in a loop shape. A first end of the core wire 21 is electrically connected to a first end of the outer winding wire 22. A second end of the core wire 21 is connected to a first terminal of an external circuit 8. A second end of the outer winding wire 22 is connected to a second terminal of the external circuit 8.

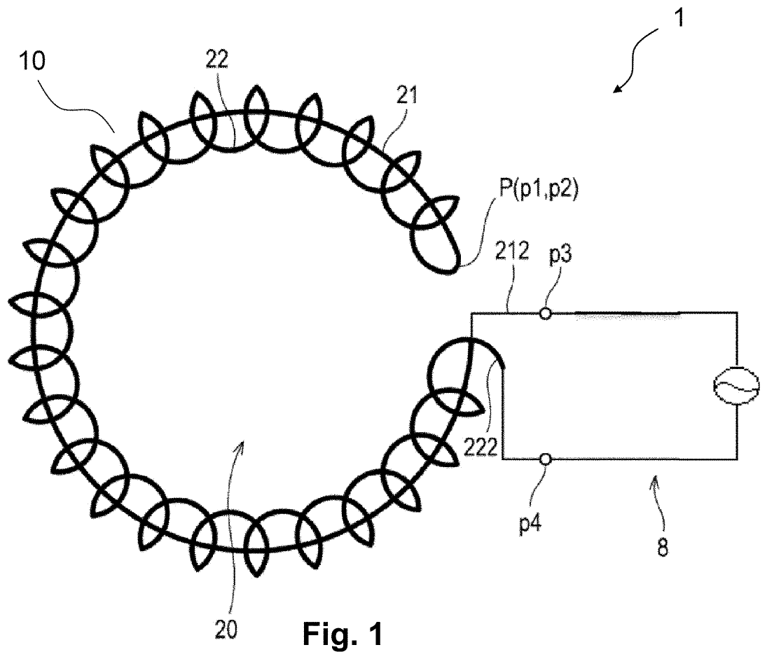

FIG. 1 shows the schematic configuration of the base wire 10 forming the electrical stimulator 1. The base wire 10 is configured with the core wire 21 and the outer winding wire 22 that is spirally wound around the core wire 21. The core wire 21 and the outer winding wire 22 are different lead wires. One end “p1” of the core wire 21 and one end “p2” of the outer winding wire 22 are connected at a point “P.” Further, the other end “p3” of the core wire 21 and the other end “p4” of the outer winding wire 22 correspond to ends of a first extension wire 212 and a second extension wire 222, respectively, that are connected to the external circuit 8, for example. Specifically, the other end “p3” is located at a different side of the core wire 21 with respect to one end “p1.” The other end “p4” is located at a different side of the outer winding wire 22 with respect to one end “p2.” The external circuit 8 is for sending an electric signal (for instance, an electric current) that is input to the core wire 21 and the outer winding wire 22. Thus, the external circuit 8 explained above works as a power supply that supplies an electric current to the base wire 10 of the electrical stimulator 1. The electrical stimulator 1 forms an electric field inside of the annulus 20 configured by winding the base wire 10. Further, the core wire 21 and the outer winding wire 22 are not limited to individual lead wires. It is also possible that the core wire 21 and the outer winding wire 22 may be formed by a single lead wire that is folded back at the point “P.”

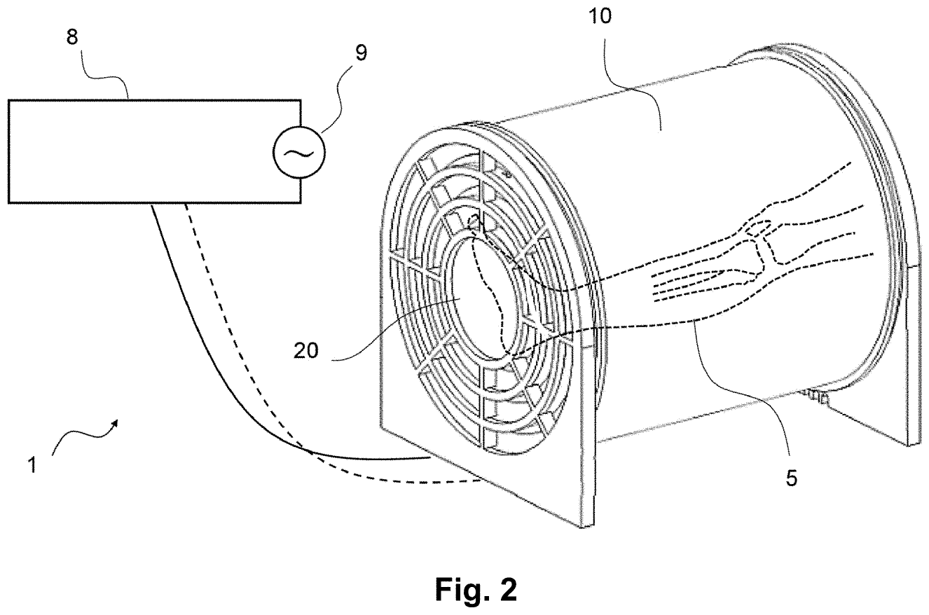

FIG. 2 is a schematic view for explaining the electrical stimulator 1 that uses the base wire 10 shown in FIG. 1. The base wire 10 is wound one or more turns on the periphery of the electrical stimulator 1, so that the annulus 20 is formed in which a living body or a part of the living body 5 (lower limb or knee joint) is held. It is preferable to form the annulus 20 by arranging the base wire 10 such that a base wire is disposed on the outer peripheral surface of an adjacent base wire without any gap therebetween. When an electric current at a predetermined frequency is supplied to the base wire 10 from an AC power supply 9 connected to the external circuit 8 with a predetermined part of the human body held in the annulus 20, an electric field is generated in the annulus 20 along the axial direction of the annulus 20 in a non-contact manner without generating a magnetic field. Further, an electric current flows from a strong electric field toward a weak electric field within the electric field in a human body part 5, such as the lower limb or the knee joint. With such a configuration, it is possible to apply a predetermined electrical stimulation to a predetermined human body part 5, such as the lower limb or the knee joint.

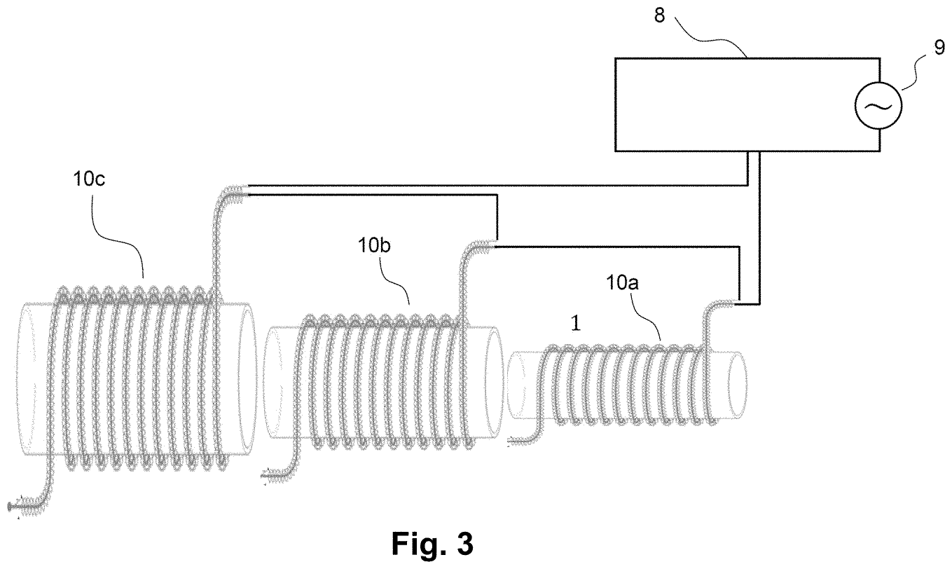

FIG. 3 is a schematic view of a vector potential generation device used in other embodiments (examples described later). In this embodiment, the base wire 10 forming the annulus 20 is configured with base wires 10a, 10b, and 10c forming a three-layered structure. Each base wire includes the core wire 21 and the outer winding wire 22, and the core wire 21 and the outer winding wire 22 are connected to each other at one end of the base wire 10. At the other end of the base wire 10, the outer winding wire 22 of the base wire 10a is connected to the core wire 21 of the base wire 10b, for example. In the same manner, the base wire 10b is connected to the base wire 10c, and an electric current at a predetermined frequency is applied to the return wire of the base wire 10a and to the outer winding wire 22 of the base wire 10c from the AC power supply 9.

A voltage (intensity of electric field) generated in the annulus 20 can be calculated based on the differential value of an electric current applied to the base wires 10a, 10b, and 10c. As described in International Publication Number WO2015/099147 in detail, basically, this calculation formula can be obtained by the winding density of the base wire, the diameter of a coil, and the like. For example, a voltage generated in the annulus 20 can be obtained based on the following equation (12) described in International Publication Number WO2015/099147. The entire contents described in International Publication Number WO2015/099147 are incorporated herein by reference.

V₂=μ₀nN₁Sω(\sqrt{a²+L²}−a)Iₘ cos(ωt) (12)

In this equation, “V₂” is the voltage obtained by accumulating an electric field E by a vector potential, “μ₀” is permeability of vacuum, “n” is the number of windings of the outer winding wire per unit length of the core wire, “N₁” is the number of windings of the base wire per unit length, “S” is the cross-sectional area of the base wire, “a” is the inner radius of the annulus, “L” is the length of the base wires 10a, 10b, and 10c, “Im” is the amplitude of an electric current, “ω” is the frequency, and “t” is the time period. Accordingly, it is possible to control a voltage generated in the annulus 20 to a desired value by controlling the structure of the electrical stimulator, for example, by controlling the length of a coil formed by winding the base wire into a loop shape, the diameter of the coil, the number of windings, and by controlling the frequency and the amplitude value of an electric current applied from the AC power supply 9.

Further, the external circuit 8 can also provide the same current or different currents at the same time for not only a single annulus 20 but also a plurality of annuluses 20 that are attached to a plurality of affected parts. Since the miniaturization of the external circuit 8 can be achieved, the external circuit can also be a module or a device that is driven by a battery. As a result, the portability of the electrical stimulator 1 further increases.

It is preferred that the external circuit 8 has a control unit that can control the parameters, such as an amount of an electric current flowing in the core wire 21 and the outer winding wire 22 of the annulus 20, a period of time for flowing the electric current, or a frequency of the electric current. In addition, it is further preferred that the control unit also has other functions. Specifically, the control unit can control the plurality of annuluses 20 at the same time and can modify the parameters such as the electric current and/or the frequency based on the data that is fed back from other sensors such as a body temperature sensor and/or a bioelectric current sensor.

### <Method for Operating Electrical Stimulator>

One aspect of the present disclosure is the above-mentioned method for operating an electrical stimulator. This method is characterized by including holding a living body or a part of the living body in an annulus, and generating an alternating current by an external circuit for a therapeutically effective time period to apply an electrical stimulation to the living body or the part of the living body. In this embodiment, the term “living body” is not limited to the human body, but includes organisms in general, such as animals. Further, the term “hold” means keeping the position of the living body or the part of the living body in the annulus. In this embodiment, the term “hold” also includes not only keeping the position of the living body or the part of the living body in the annulus by fixing the living body or the part of the living body by a fixture, but also keeping the position of the living body or the part of the living body in the annulus by accommodating the living body or the part of the living body on a surface having a recessed shape or a recessed curved shape, and keeping the position of the living body or the part of the living body in the annulus by placing the living body or the part of the living body on a flat surface, for example. In a preferred embodiment, a flat mounting table or the like may be provided in the annulus.

It is preferable to use, as a material for forming the mounting table, an insulating material through which an electric current is prevented from flowing. It is further preferable to use a resin material, such as rubber, polyethylene, or polyvinyl chloride, as a material for forming the mounting table. From the viewpoint of heat resistance, it is also possible to use ceramic as a material, for example.

An electric current that is generated in the external circuit to apply an electrical stimulation to the living body or the part of the living body held in the annulus may be a continuous alternating current or a pulsed alternating current. The employed frequency can be in a range between a few Hz and a few kHz according to the individual or the condition of the damage or injuries. To perform treatment on the articular cartilage, for example, a frequency of an alternating current is preferably 10 to 50 kHz, and is more preferably approximately 20 kHz. By controlling the structure of the vector potential generation device and an electric current applied to the vector potential generation device, it is possible to control the intensity of the electric field generated in the annulus. This intensity of the electric field can be suitably adjusted according to the part of the living body being the treatment target or the symptoms. Although not particularly limited, the intensity of the electric field in the annulus is preferably approximately 0.1 to 1 V/m, and is more preferably 0.17 to 0.27 V/m. It is further preferred that the intensity of the electric field in the annulus is approximately 0.22 V/m. In this case, the intensity of an electrical stimulation applied to the living body held in the annulus can be estimated, as the value of the electric current flowing through the living body, from the intensity of the electric field applied to the electrical stimulator and the impedance of the living body held in the annulus, for example.

In some examples, the therapeutically effective time period is the time period during which the electrical stimulator of this embodiment is operated to reduce or eliminate one or more signs or symptoms of the disease, or the disorder described herein. For example, the therapeutically effective time period is at least 30 minutes, 60 minutes, or 90 minutes per day. It is preferable to operate the electrical stimulator continuously or discontinuously every day one, two, or three times per day, five or more days per week for one to three weeks or more. This operating time period is given for the sake of example and is not restrictive. A plan for additional treatment may include other therapies based on symptom of a disease or injury to be treated or based on lifestyle.

<Treatment Method that Uses Electrical Stimulator>

Another aspect of the present disclosure provides a method for treating a living body using the above-mentioned electrical stimulator and includes the following embodiments.

(1) A method for treating a living body using an electrical stimulator, the electrical stimulator including a base wire configured with a core wire having an insulating film, and an outer winding wire wound around the core wire with the core wire serving as a winding axis, wherein an annulus is formed by winding the base wire in a loop shape, a first end of the core wire is electrically connected to a first end of the outer winding wire, a second end of the core wire is connected to a first terminal of an external circuit, and a second end of the outer winding wire is connected to a second terminal of the external circuit, the method including: holding a living body or a part of the living body of a subject in the annulus; and generating an alternating current in the external circuit for a therapeutically effective time period to apply an electrical stimulation to the living body or the part of the living body.
(2) The method of (1), in which the part of the living body is a stem cell, or a joint or a ligament of the subject in need of treatment.
(3) The method of (1), in which the part of the living body is a knee joint of a subject in need of treatment for osteoarthritis of the knee.
(4) The method of (1), in which the frequency of the alternating current is 10 to 50 kHz.
(5) The method of (1), in which the frequency of the alternating current is approximately 20 kHz.
(6) The method of (1), in which the therapeutically effective time period is at least 30 minutes/day.
(7) The method of (1), in which the alternating current is applied by the external circuit such that the intensity of an electric field in the annulus is 0.17 to 0.27 V/m.
(8) The method of (1), in which the alternating current is applied by the external circuit such that the intensity of an electric field in the annulus is approximately 0.22 V/m.
(9) The treatment method of (1), in which the annulus is formed in a concentric shape of multiple layers.
(10) A method of treating a disorder related to bone or joint in a patient, using an electrical stimulator, the electrical stimulator comprising a base wire configured with a core wire having an insulating film, and an outer winding wire wound around the core wire with the core wire serving as a winding axis; wherein an annulus is formed by winding the base wire in a loop shape, a first end of the core wire is electrically connected to a first end of the outer winding wire, a second end of the core wire is connected to a first terminal of an external circuit, and a second end of the outer winding wire is connected to a second terminal of the external circuit, the method comprising the steps of holding the living body or a part of the living body of a subject in the annulus, and generating an alternating current in the external circuit for a therapeutically effective time period to apply an electrical stimulation to the living body or the part of the living body.
(11) The method of (10), in which the disorder related to bone or joint is selected from the group consisting of chronic osteoarthritis, rheumatoid arthritis, reactive arthritis, rotator cuff injuries, plantar fasciitis, spondylolisthesis, and ligamentous injuries.
(12) The method of (10), in which the disorder related to bone or joint is a disorder related to articular cartilage.

Although a disease or injury to be treated by the treatment method of the present disclosure is not particularly limited, examples of a disease or injury to be preferably treated by the treatment method of the present disclosure include diseases related to the bone or the joint. For example, the treatment method of the present disclosure may be used for treating rheumatoid arthritis, fibrodysplasia ossificans progressiva (FOP), diffuse idiopathic skeletal hyperostosis (DISH), ankylosing spondylitis, or a wide range of diseases involving overactive or improper bone growth, such as heterotopic ossification. The treatment method of the present disclosure may be used to remove a bone lump for treating a disease involving neoplastic bone formation or bone tumor, such as osteosarcoma, chondrosarcoma, Ewing's sarcoma, osteoblastoma, or osteoid osteoma.

In the same manner, the treatment method of the present disclosure may be used to treat the disorders such as chronic osteoarthritis, rheumatoid arthritis, reactive arthritis, rotator cuff injury, planter fasciitis, spondylosis, and/or spinal stenosis, as well as to remove bone spurs formed in the leg, the shoulder, the neck, the spine or the like (that is, “bone spur”) as the result thereof.

An example of another disease or injury to be preferably treated includes ligament injury. The joints of the body are supported by the ligaments. The ligament is a tough band being a connective tissue that binds one bone to another bone. A sprain is a simple stretch or tear of the ligament. Regions where sprain occurs most easily are the ankle, the knee, and the wrist. The lightest sprain may be cured by rest, ice cooling treatment, compression treatment, elevation, exercise and/or a physical therapy. A moderate sprain may require a fixing period. A heavy sprain may require surgery to restore the torn ligament.

Examples of other diseases or injuries to be treated include diabetes, gastritis, peptic ulcer, ulcerative colitis, irritable colon, hemorrhoid; bronchial asthma including cold, tonsillitis, sinusitis, and chronic bronchitis; cardiovascular disease including phlebitis, endarteritis, and varix; and mental disorder, such as depression, aggression, anxiety, and stress. The examples of other diseases or injuries to be treated further include Parkinson's disease, epilepsy, migraine, cerebral apoplexy, Alzheimer, and other degenerative brain disorders, and also include encephalopathy and mental disorder including cerebral palsy, mental retardation, hyperactivity, and learning disabilities. In addition to the above, the treatment method of the present disclosure may also be used to treat the genitourinary system of women, such as irregular menstruation, sterility, endometritis, and endometriosis, and of men, such as orchitis, prostatitis, and oligospermia.

Advantageous effects of the treatment method of the present disclosure are considered as follows. In the central nervous system, for example, neurochemicals necessary for transmitting impulses or instructions are synthesized at the synaptic level, thus improving the electric activity of the cells of the central nervous system and hence, it is possible to increase efficiency of the brain cells. Another advantageous effect is considered as follows. The treatment method of the present disclosure has an ability to stabilize genes and prevent the activity of oxygen free radicals forming in cells, thus being useful for delaying the aging process. Advantageous effects in treating the articular cartilage will be described in detail in examples which will be described later.

When VP treatment is performed on the epigastric region as prophylactic treatment before surgery, blood perfusion to the body and extremities is increased and hence, it is possible to reduce inflammatory response of a damage. It is also shown that performing VP treatment on the surgical site before surgery also accelerates the healing of the surgical site. In addition to the above, the VP treatment can also reduce or alleviate symptoms of postoperative nausea, motion sickness, or nausea of other causes, such as vomiting.

In another embodiment, the treatment method of the present disclosure may be used as auxiliary means for another treatment including at least one of cell implant, a cultured skeleton, and a growth factor, or to treat cartilage defect and to prevent tumor metastasis.

In another embodiment, in regenerative medicine where angiogenesis is performed by stem cells derived from the bone marrow, for example, to treat angina pectoris, myocardial infarction or the like, the treatment method of the present disclosure may be applicable to a cell storage/culture apparatus that can efficiently culture a large volume of stem cells in an artificial environment to propagate target cells or an organ from stem cells or the like in a culture dish and to implant the cells or the organ into a human.

In another embodiment, the treatment method of the present disclosure may be used to increase a bone density in adjusting a bone receiving a dental or orthopedic implant, or to treat a bone gap, such as a defective part of the alveolar bone, adjacent parts of the alveolar bone, or a defective bone part caused by surgery, external injury, or disease.

In another embodiment, the treatment method of the present disclosure may also be used for similar purposes in non-human mammals in the veterinary field for mammals other than humans, for example, for companion animals, such as dogs or cats, or horses, particularly racehorses.

Next, the present invention will be explained in more detail by giving examples. However, the present invention is not limited by these examples.

### EXAMPLE

### <Experimental Device>

FIG. 3 is a schematic view of the vector potential generation device (hereinafter referred to as “VP device”) used in the following examples. As shown in FIG. 3, three base wires (VP wires) 10a, 10b, and 10c are wound around annuluses (annular members). The three base wires have the same length of 225 mm. The annuluses have different diameters of 130 mm, 170 mm, and 210 mm. The number of windings is 97 T. A winding density of a winding wire is 950 T/m. The three base wires are concentrically arranged when finally assembled. The three base wires forming this VP device are connected in series on the circuit and hence, the VP device actually corresponds to a three-layered VP device. The device has a length of approximately 30 cm. When a sine wave of 10.8 App is applied to VP coils, the electric field has an intensity of approximately 0.22 V/m in the longitudinal direction and a voltage of approximately 67 mV is applied to both ends of the annuluses.

### (Example 1) Effects of Electrical Stimulation on Structural Changes in Rat Tibial Articular Cartilage by Hindlimb Suspension

In this example, rats with tails suspended were used to morphologically compare and investigate structural changes in the tibial articular cartilage when a VP electrical stimulation was applied for different time periods of one to three weeks.

### <Experimental Method and Materials>

Seventy-two Wistar strain male rats of seven weeks old were used, and were randomly classified as follows.

Tail suspension group (HS group): Tails of rats were suspended for one, two, or three weeks.

Electrical stimulation group (VP group): Tails of rats were suspended for one, two, or three weeks, and the rats were energized at an alternating current (20 kHz) under the above-mentioned conditions for energization by the vector potential generation device (VP device) for 30 minutes/day and 5 days/week under anesthesia. In this case, assuming that a voltage of approximately 67 mV is generated at both ends of the VP device and the impedance of the rat held in the device is 500Ω, it is estimated that an electric current of 0.13 mA flows.

Control group (CO group): Rats were normally bred in cages for one, two, or three weeks.

After the end of the experimental periods, the rats of each group were euthanized and, thereafter, the tibias were excised and histologically observed.

### <Preparation of Non-Decalcified Resin-Embedded Ground Sections>

After the end of an electrical stimulation experiment, each rat was euthanized with carbon dioxide gas, the skin was peeled off and the soft tissue was removed to excise the tibia. The proximal part of the tibia was cut sagittally by a hand motor (Labo Force made by YOSHIDA DENTAL TRADE DISTRIBUTION CO., LTD.) equipped with a diamond disk (Meisinger made by GC Corporation) and was promptly immersed in a fixative overnight. After each sample was washed with water, the sample was dehydrated with alcohol series. The sample was cleaned with acetone and, thereafter, was embedded in Rigolac resin, and was heat-polymerized in a thermostatic oven (DY300 made by Yamato Scientific Co., Ltd.). A block was trimmed by a band saw (K-100 made by HOZAN TOOL IND. CO., LTD.), and then roughly ground by a model trimmer (made by YOSHIDA DENTAL TRADE DISTRIBUTION CO., LTD.). The block was ground to have a thickness of approximately 150 μm by grinding wheels of three stages (a rough grinding stone, a medium grinding stone, and a finishing grinding stone), and was then carefully ground with a dedicated film to remove scratches on the surface. The ground surface was etched with 0.1M hydrochloric acid and, thereafter, was stained with a heated 1% toluidine blue solution. The ground section was photographed by a light microscope (BX53-33-FL-2 made by Olympus Corporation) with a photographing device (DP73-SET-B made by Olympus Corporation).

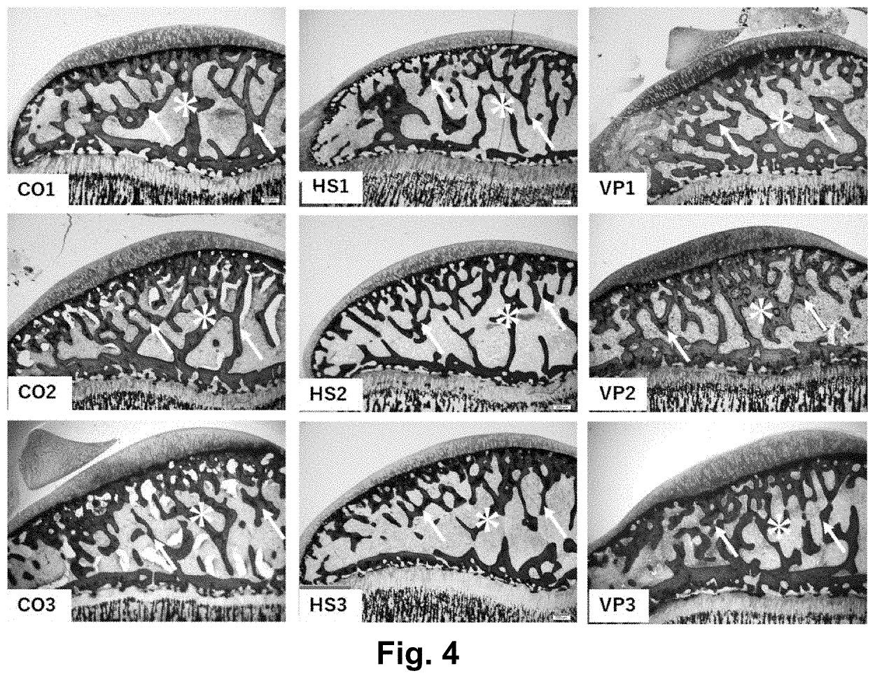

The results are shown in FIG. 4. FIG. 4 shows low-magnified images of the proximal epiphysis of the tibia after one, two, and three weeks in each group. For example, “CO1” means the control group after one week, and “CO2” means the control group after two weeks. Further, “VP1” means the electrical stimulation group after one week from the start of treatment, and “VP2” means the electrical stimulation group after two weeks from the start of treatment. Each asterisk denotes the cancellous bone of the epiphysis, and arrows denote the trabecular bone forming the cancellous bone. In FIG. 4, each bar represents 500 μm. In the CO group, the trabecular bone of the cancellous bone of the epiphysis is densely present. However, in the HS group, the trabecular bone is thin, thus having a lower density as a whole. Such a difference between both groups became conspicuous with the progress of the experimental period. In the HS group, the articular cartilage located on the surface of the epiphysis has a thickness smaller than that of the CO group. Such a difference was started to be observed after one week from the start of the experiment, and a similar condition was observed throughout the experimental period of three weeks. In contrast, in the VP group, the thickness and the density of the trabecular bone of the cancellous bone of the epiphysis were close to those of the CO group throughout the experimental period, that is, a reduction in thickness and density by a decrease in loading was suppressed. In the same manner as the HS group, the rats in the VP group were in a loading decreased state during the experimental period. However, the thickness of the articular cartilage in the VP group was close to that of the CO group.

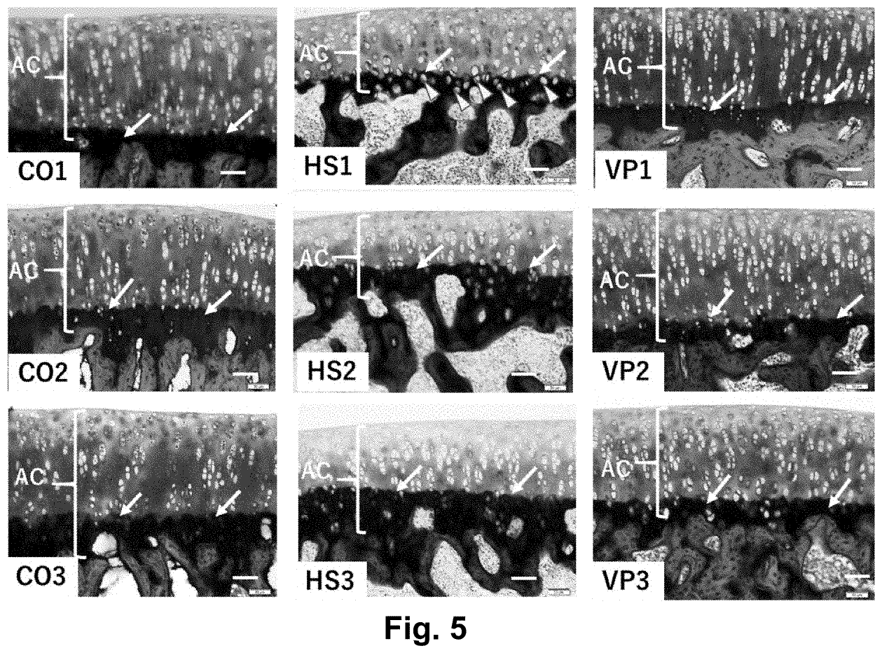

FIG. 5 shows enlarged images of the articular cartilages shown in FIG. 4, and shows a comparison of the thickness of the entire articular cartilage and the thickness of a calcified layer at the anteroposterior mid-portion of the epiphysis. In FIG. 5, each bar represents a length of 50 μm, and arrows show a height of a tide mark (boundary face between deep and calcified layers) and calcified layer was stained in navy blue with a toluidine blue dye. Symbol “AC” denotes the articular cartilage and the thickness of the articular cartilage. When a comparison was made on the thickness of the entire articular cartilage at the anteroposterior mid-portion where the tibia and the femur are brought into contact with each other most firmly, in the HS group, the thickness of the articular cartilage reduced after one week from the start of the experiment. In contrast, in the VP group, the thickness of the articular cartilage was maintained after one week.

The calcified layer stained in navy blue is located at the lower portion of the articular cartilage. In the CO group and the VP group, the white chondrocytes are rarely observed in the calcified layers at any time. However, in the HS group, a large number of large white chondrocytes are present as shown by arrowheads in HS1 of FIG. 5 (FIG. 5). This is caused by a rapid upward expansion of the calcified layer in navy blue due to the effect of the decrease in loading on the articular cartilage by suspension of the tail. Such an expansion of the calcified layer causes the cells of an uncalcified layer at the upper portion of the articular cartilage to be embedded in the calcified layer, so that the large number of large white chondrocytes are generated. Due to such an expansion of the calcified layer, the thickness of the uncalcified layer of the articular cartilage reduces in the HS group. In contrast, in the VP group, calcification is suppressed and hence, the thickness of an uncalcified layer of the articular cartilage is maintained. In the CO group, the bone stained in purple is present at the lower portion of the calcified layer. However, in the HS group, the bone is not formed due to a decrease in loading, and the lower portion of the calcified layer of the articular cartilage is absorbed. In the VP group, the bone at the lower portion of the articular cartilage was maintained, and the articular cartilage and the bone at the lower portion of the articular cartilage were in states close to those of the CO group.

### <Morphometry of Articular Cartilage>

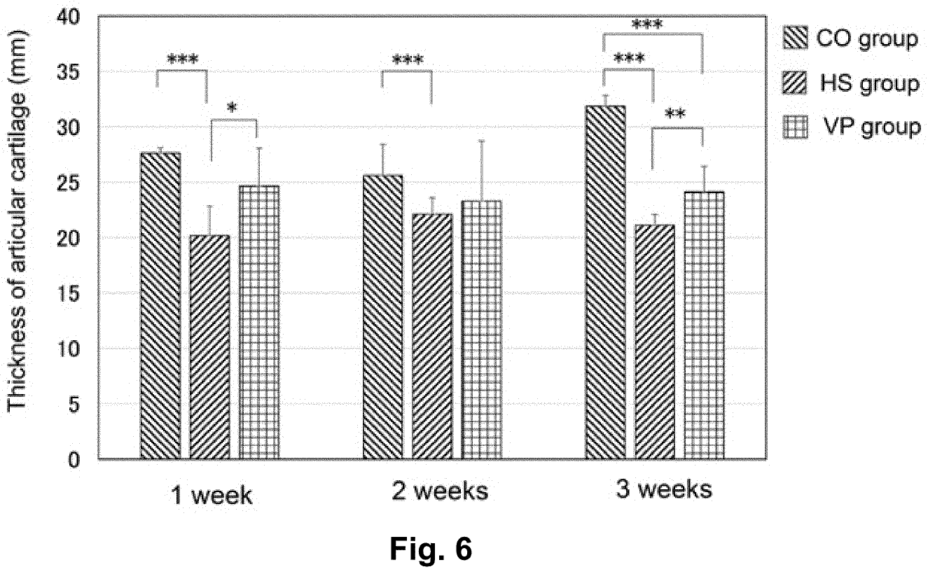

Sections of the above-mentioned non-decalcified resin-embedded ground section was used to measure the thickness and the area of the articular cartilage by an interlocking manual measurement system (WinRoof made by MITANI CORPORATION). The results are shown in FIG. 6. As shown in FIG. 6, after the start of the experiment, the thickness of the articular cartilage reduced more significantly in the HS group than in the CO group for all periods. In the samples of the VP group after one week and three weeks from the start of the experiment, the thickness of the articular cartilage significantly (P<0.05) larger than that of the HS group was maintained. In FIG. 6, the results of a significant difference test by a Turkey test are shown by *: P<0.05, **: P<0.01, and ***: P<0.001.

### <Preparation of Decalcified Paraffin Sections (Immunostaining and Safranin-O Staining)>

Samples were dehydrated with alcohol by a method substantially the same as the method adopted for preparing the non-decalcified resin-embedded ground sections. Each samples was cleaned with benzene and, thereafter, was embedded in a paraffin to prepare a block. The block was trimmed by a knife and was attached to a wooden stand. Sections with a thickness of 4 μm were cut by a microtome (YAMATO KOHKI INDUSTRIAL CO., LTD, Litratome), and immunostaining and safranin-O staining were performed on the sections. The sections were photographed and observed by an light microscope (BX53-33-FL-2 made by Olympus Corporation) with a photographing device (DP73-SET-B made by Olympus Corporation).

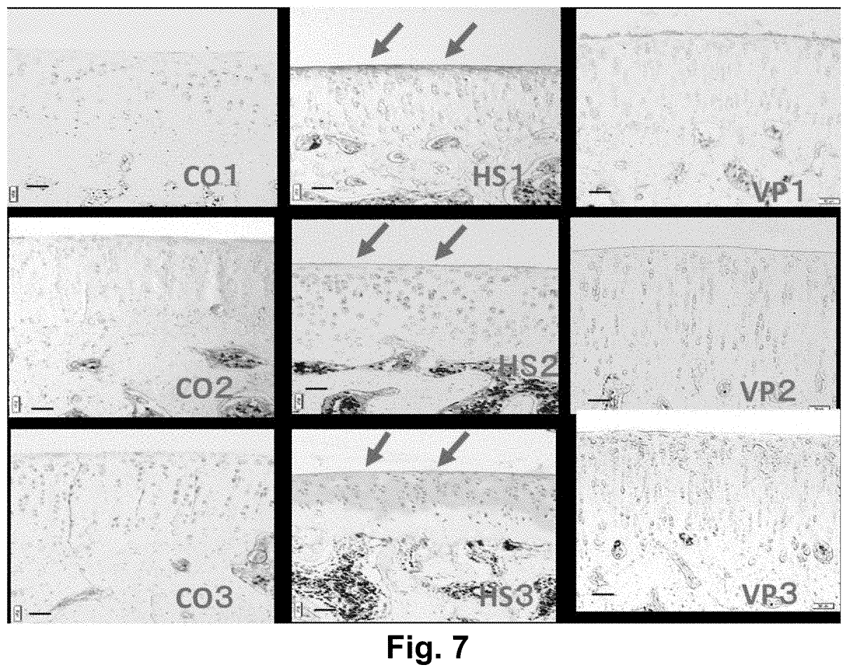

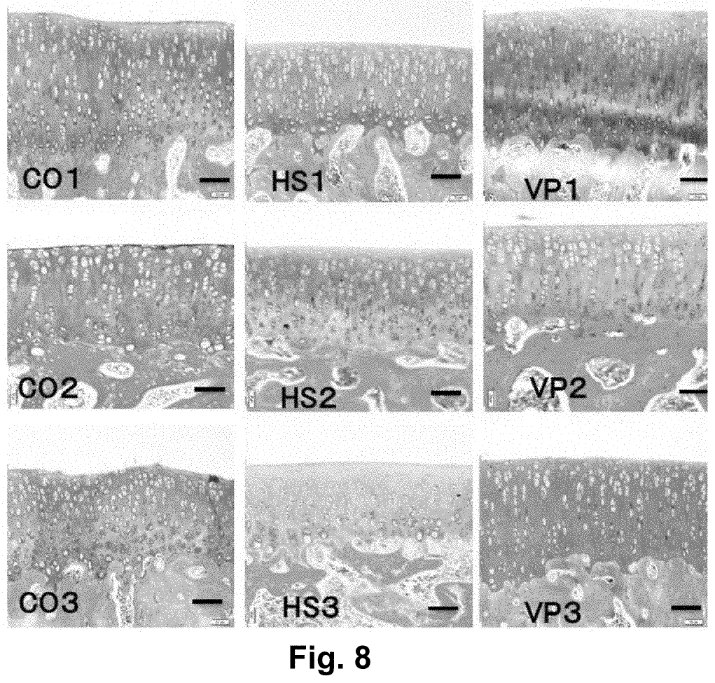

The results are shown in FIG. 7 and FIG. 8. FIG. 7 shows a comparison of the results of the immunostaining of the articular cartilage with matrix metalloproteinase-3 (MMP-3) between the respective groups each including three samples. In FIG. 7, each bar represents a length of 50 μm, and arrows show parts having a positive reaction of MMP-3. In HS1 and HS2, a reaction of MMP3 was observed on the surface of the articular cartilage. In HS3, a reaction was expanded to the deep portion of the articular cartilage. In the CO group and the VP group, no reaction of MMP3 was observed for all experimental periods. MMP3 is an enzyme that degrades organic compounds of the cartilage. As can be understood from these results, destruction of the articular cartilage progressed in the HS group. However, the articular cartilage was maintained in the VP group.

FIG. 8 shows a comparison of safranin-O stainability in the articular cartilage between the respective groups each including three samples. In FIG. 8, each bar represents a length of 50 μm. In the HS group, safranin-O stainability in the articular cartilage is reduced after one week from the start of the experiment, and the reduction progressed thereafter. In contrast, in the VP group, although stainability was reduced in VP2, stainability in each of VP1 and VP3 was substantially equal to or higher than that of CO1 and CO3. Safranin has a high affinity for organic compounds (proteoglycans) of the articular cartilage. From safranin stainability, a possibility was suggested that although organic compounds are reduced in the HS group, organic compounds are maintained in the VP group.

### (Example 2) Effects of Different Time Periods of Interventions with Electrical Stimulation on Structural Changes in Rat Tibial Articular Cartilage by Decrease in Loading

This example aims to morphologically compare and investigate effects of different time periods of interventions with a non-contact electrical stimulation on structural changes in the rat tibial articular cartilage by hindlimb suspension.

### <Experimental Method and Materials>

Seventy-two Wistar strain male rats of seven weeks old were used, and were randomly classified as follows.

Control group (CO group): Rats were normally bred in cages for three weeks.

Hindlimb suspension group (HS group): The hindlimbs of rats were suspended for three weeks.

Electrical stimulation group (VP group): The hindlimbs of rats were suspended for three weeks, and an electrical stimulation was applied, by using the VP device, to the rats for 15 minutes, 30 minutes, 60 minutes, and 90 minutes/day, 5 days/week, for three weeks. Assuming that conditions for energization are the same as that in the example 1, a voltage of approximately 67 mV is generated at both ends of the VP device, and the impedance of the rat held in the device is 500Ω, it is estimated that an electric current of 0.13 mA flows.

### <Preparation of Non-Decalcified Resin-Embedded Ground Samples>

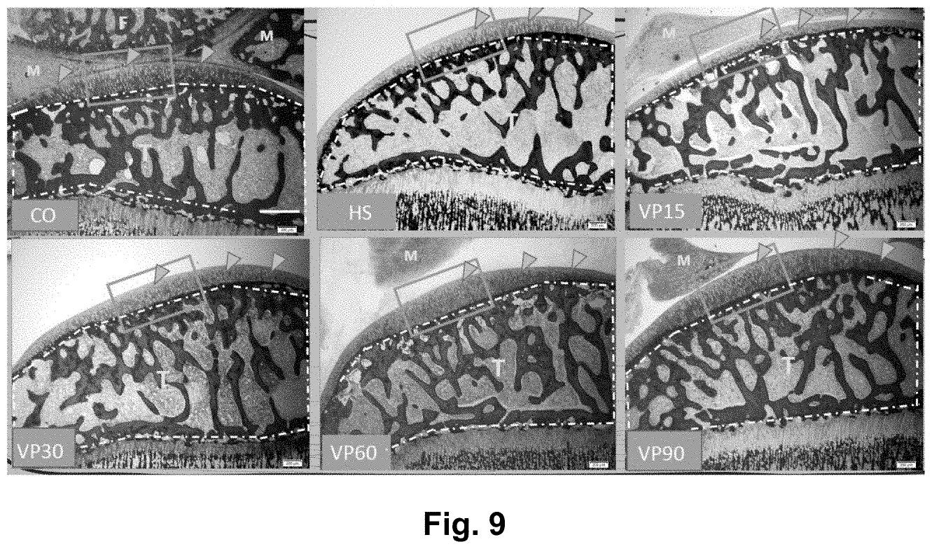

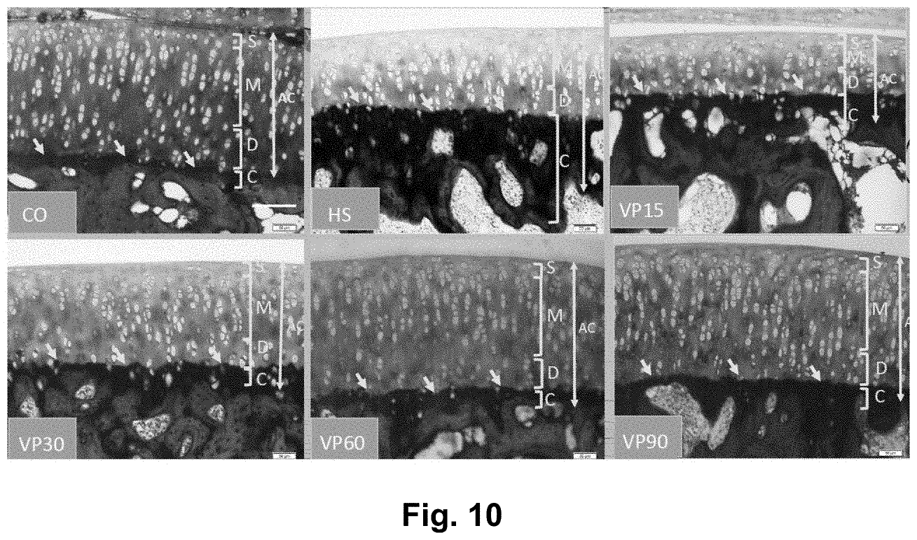

Non-decalcified resin-embedded ground sections were prepared by a method substantially the same as that in the example 1, and toluidine blue staining was performed on the non-decalcified resin-embedded ground sections. The results are shown in FIG. 9 and FIG. 10. In FIG. 9, each bar represents a length of 200 μm, “F” denotes the femur, “T” denotes the tibia, “M” denotes the meniscus, and “arrowhead” denotes the tibial articular cartilage. In FIG. 10 obtained by enlarging portions each surrounded by a rectangle in FIG. 9, each bar represents a length of 50 μm, arrows denote the position of a tide mark, “AC” denotes the entire articular cartilage, “S” denotes a shallow layer, “M” denotes a middle layer, “D” denotes a deep layer, and “C” denotes a calcified layer. In FIG. 9 and FIG. 10, VP15 indicates a sample to which an electrical stimulation is applied for 15 minutes. In the cross section of the articular cartilage stained in blue or purple in FIG. 10, a large number of white dots are chondrocytes. Portions other than the chondrocytes indicates “matrix.” The matrix includes collagen fibers and proteoglycans that fills gaps between the collagen fibers. Proteoglycans are contained in the portions of the matrix stained color of blue or purple. It can be understood that the amount of proteoglycan in the articular cartilage is increased more in VP30 to VP90 compared with HS.

### <Preparation of Scanning Electron Microscope Samples>

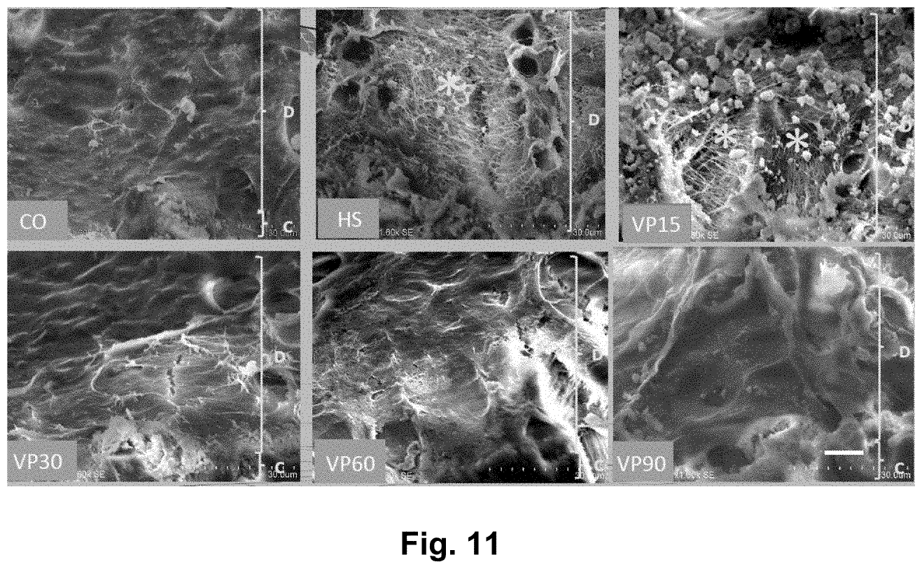

Samples were dehydrated with alcohol by a method substantially the same as the method adopted for preparing the non-decalcified resin-embedded ground sections. Each sample was immersed in t-butyl and, thereafter, was frozen in a refrigerator and was dried by a vacuum freeze dryer (ES-2030 made by Hitachi, Ltd.). The sample was mounted on a sample stand, and a non-conductive adhesive agent (DOTITE) was applied to the sample stand by coating. Carbon and platinum are vacuum-deposited on the surface of the sample respectively by a carbon coater (VC-100 made by VACUUM DEVICE) and by an ion sputter (E-1010 made by Hitachi, Ltd.), and the sample was observed by a scanning electron microscope (S-3400 made by Hitachi, Ltd.). The results are shown in FIG. 11. In FIG. 11, each bar represents a length of 10 μm, arrows denote the position of a tide mark, “*” denotes a region where the fibers of the matrix are clearly observed by treatment with sodium hypochlorite, “D” denotes a deep layer, and “C” denotes a calcified layer. In FIG. 11, the presence of thin collagen fibers is observed in the vicinity of “*” in HS and VP15. However, in CO and VP30 to VP90, the presence of fibers is not apparent. The reason is that proteoglycans are densely present between the fibers, and the proteoglycans cover the fibers. In preparing these samples, organic compounds (proteoglycans) were slightly eluded on the cross section of the articular cartilage with sodium hypochlorite. Despite such elusion, a large number of organic compounds remains in CO and VP30 to VP90. This means that abundant proteoglycans were originally present in these samples.

The results shown in FIGS. 9 to 11 suggests the following. Although the amounts of proteoglycans and collagen fibers in the articular cartilage reduce with a decrease in mechanical loading, such reduction can be suppressed by applying an electrical stimulation by the VP generation device for more than 30 minutes.

The method for operating an electrical stimulator of the present disclosure is effectively used to treat various diseases, particularly, a disease related to the articular cartilage.

The method for operating the electrical stimulator and to the method for treating a living body using the electrical stimulator being thus described, it will be apparent that the same may be varied in many ways. Such variations are not to be regarded as a departure from the spirit and scope of the invention, and all such modifications as would be apparent to one of ordinary skill in the art are intended to be included within the scope of the following claims.

What is claimed is:

1. A method for treating a living body using an electrical stimulator,
the electrical stimulator including:
a base wire configured with a core wire having an insulating film, and an outer winding wire wound around the core wire with the core wire serving as a winding axis;
wherein an annulus is formed by winding the base wire in a loop shape,
a first end of the core wire is electrically connected to a first end of the outer winding wire,
a second end of the core wire is connected to a first terminal of an external circuit, and
a second end of the outer winding wire is connected to a second terminal of the external circuit,
the method comprising the steps of:
holding the living body or a part of the living body of a subject in the annulus; and
generating an alternating current in the external circuit for a therapeutically effective time period to apply an electrical stimulation to the living body or the part of the living body.

2. The method according to claim 1, wherein
the part of the living body is a stem cell, or a joint or a ligament of the subject in need of treatment.

3. The method according to claim 1, wherein
the part of the living body is a knee joint of the subject in need of treatment for osteoarthritis of the knee.

4. The method according to claim 1, wherein
a frequency of the alternating current is 10 to 50 kHz.

5. The method according to claim 1, wherein
a frequency of the alternating current is approximately 20 kHz.

6. The method according to claim 1, wherein
the therapeutically effective time is at least 30 minutes/day.

7. The method according to claim 1, wherein
the alternating current is applied in the external circuit so that the electric field strength in the annulus is 0.17 to 0.27 V/m.

8. The method according to claim 1, wherein
the alternating current is applied in the external circuit so that the electric field strength in the annulus is approximately 0.22 V/m.

9. The method according to claim 1, wherein
the annulus is formed in a concentric shape of multiple layers.

10. A method for treating a disorder related to bone or joint in a patient, using an electrical stimulator,
the electrical stimulator including:
a base wire configured with a core wire having an insulating film, and an outer winding wire wound around the core wire with the core wire serving as a winding axis;
wherein an annulus is formed by winding the base wire in a loop shape,
a first end of the core wire is electrically connected to a first end of the outer winding wire,
a second end of the core wire is connected to a first terminal of an external circuit, and
a second end of the outer winding wire is connected to a second terminal of the external circuit,
the method comprising the steps of:
holding the living body or a part of the living body of a subject in the annulus; and
generating an alternating current in the external circuit for a therapeutically effective time period to apply an electrical stimulation to the living body or the part of the living body.

11. The method according to claim 10, wherein
the disorder related to bone or joint is selected from the group consisting of chronic osteoarthritis, rheumatoid arthritis, reactive arthritis, rotator cuff injuries, plantar fasciitis, spondylosis, and ligamentous injuries.

12. The method according to claim 10, wherein
the disorder related to bone or joint is a disorder related to articular cartilage.
